(19) United States
(12) Patent Application Publication
(10) Pub. No.: US 2026/0248821 A1
(43) Pub. Date: Aug. 27, 2026

(54) CANNABIS PLANT MATERIAL AS AN AGENT FOR MITIGATING METABOLIC SYNDROME

(71) Applicant: University of South Carolina, Columbia, SC (US)

(72) Inventors: Elizabeth Murphy, Columbia, SC (US); Daping Fan, Columbia, SC (US); Kandy T. Velazquez, Columbia, SC (US); Reilly Enos, Columbia, SC (US); Brandon VanderVeen, Columbia, SC (US)

(73) Assignee: University of South Carolina, Columbia, SC (US)

(21) Appl. No.: 19/062,842

(22) Filed: Feb. 25, 2025

Related U.S. Application Data
(60) Provisional application No. 63/615,357, filed on Dec. 28, 2023.

(51) Int. Cl. A61K 31/00; A61P 3/04

(52) U.S. Cl. CPC A61K 31/658; A61P 3/04

(57) ABSTRACT

Described herein are methods of treatment of metabolic syndrome using Cannabis Plant Material to reduce fasting glucose and insulin and lower the homeostasis model assessment-estimated insulin resistance (HOMA-IR) index to mitigate metabolic syndrome and associated Type II Diabetes, Coronary Heart Disease, and Stroke, pharmacological compounds for same and methods for making the pharmacological compounds.

### TECHNICAL FIELD

The subject matter disclosed herein is generally directed to methods of treatment of metabolic syndrome using Cannabis Plant Material to reduce fasting glucose and insulin and lower the homeostasis model assessment-estimated insulin resistance (HOMA-IR) index to mitigate metabolic syndrome and associated Type II Diabetes, Coronary Heart Disease, and Stroke, pharmacological compounds for same, and methods for making the pharmacological compounds.

### BACKGROUND

Metabolic syndrome is a collection of cardiometabolic risk factors that includes obesity, insulin resistance, hypertension, and dyslipidemia. Although there has been significant debate regarding the criteria and concept of the syndrome, this clustering of risk factors is unequivocally linked to an increased risk of developing type II diabetes and cardiovascular disease. Based on current population estimates, nearly 100 million (approximately 1 in 3 individuals) have metabolic syndrome in the United States. It is often characterized by insulin resistance. Indeed, a symptom of metabolic syndrome is insulin resistance, defined as the inability of insulin to optimally stimulate the transport of glucose into the body's cell (hyperinsulinemia or impaired glucose tolerance). This can lead to type II Diabetes and Cardiovascular Disease. Estimated cost of diagnosed diabetes is $327 billion in medical costs and lost productivity annually in the US. Heart Disease and Stroke costs our health care system $216 billion per year. Obesity costs the US health care system nearly $173 billion a year. Combined this is over $700 billion/year. While there are a number of medications on the market to improve insulin sensitivity in individuals with metabolic syndrome, these medications come with side effects, are expensive, and are not always effective.

Obesity and its attendant metabolic disorders represent a major public health challenge. In 2017-2018, the age-adjusted prevalence of obesity in adults in the US was 42.4%, and there were no significant differences between men and women among all adults or by age group. The age-adjusted prevalence of severe obesity in adults was 9.2% and was higher in women than in men. The mortality burden associated with these figures is staggering; 300,000-400,000 deaths per year in the US are directly attributable to obesity. Further, it increases risk for a plethora of chronic diseases including diabetes, cancer, cardiovascular disease, steatohepatitis, and neurodegenerative diseases. It has been predicted that the unabated increase in obesity in the US would lead to a plateau or even a decrease in life expectancy in the first half of the 21st century.

Accordingly, it is an object of the present disclosure to provide methods of treatment of metabolic syndrome using Cannabis Plant Material to reduce fasting glucose and insulin and lower the homeostasis model assessment-estimated insulin resistance (HOMA-IR) index to mitigate metabolic syndrome and associated Type II Diabetes, Coronary Heart Disease, and Stroke.

Citation or identification of any document in this application is not an admission that such a document is available as prior art to the present disclosure.

### SUMMARY

The above objectives are accomplished according to the present disclosure by providing in one instance a therapeutic method for treating metabolic syndrome. The therapeutic methods may include administering an effective dose of High Delta (9)-tetrahydrocannabinol (THC) Cannabis Plant Material to a subject; wherein the effective dose reduces a fasting glucose level and a fasting insulin level and lowers a homeostasis model assessment-estimated insulin resistance (HOMA-IR) index in the subject; and wherein the effective dose of High Delta (9)-tetrahydrocannabinol (THC) Cannabis Plant Material increase insulin sensitivity in the subject. Further, the effective dose of High Delta (9)-tetrahydrocannabinol (THC) Cannabis Plant Material may be administered to the subject in an increasing dosage. Still, the effective dose of High Delta (9)-tetrahydrocannabinol (THC) Cannabis Plant Material may be administered in a dosage of 30 to 40 mg. Moreover, the effective dose of High Delta (9)-tetrahydrocannabinol (THC) Cannabis Plant Material may be administered daily. Again, the effective dose of High Delta (9)-tetrahydrocannabinol (THC) Cannabis Plant Material may be administered to a subject on a high fat diet. Still yet, the high fat diet may comprise at least 35% of the subject's total daily calories comprise fat. Further again, the effective dose of High Delta (9)-tetrahydrocannabinol (THC) Cannabis Plant Material lowers may include an amount of at least one CD11b+Ly6g+ monocyte in a subject. Furthermore, the effective dose of High Delta (9)-tetrahydrocannabinol (THC) Cannabis Plant Material may lower an amount of at least one CD11c+MHCII+ M1 pro-inflammatory macrophages in a subject.

In a further embodiment, the current disclosure provides a treatment for reducing obesity and associated metabolic perturbations. The method may include administering an effective dose of High Delta (9)-tetrahydrocannabinol (THC) Cannabis Plant Material to a subject; wherein the effective dose reduces a fasting glucose level and a fasting insulin level and lowers a homeostasis model assessment-estimated insulin resistance (HOMA-IR) index in the subject; and wherein the effective dose of High Delta (9)-tetrahydrocannabinol (THC) Cannabis Plant Material increase insulin sensitivity in the subject. Further, the effective dose of High Delta (9)-tetrahydrocannabinol (THC) Cannabis Plant Material may be administered to the subject in an increasing dosage. Still further, the effective dose of High Delta (9)-tetrahydrocannabinol (THC) Cannabis Plant Material is administered in a dosage of 30 to 40 mg. Again still, the effective dose of High Delta (9)-tetrahydrocannabinol (THC) Cannabis Plant Material may be administered daily. Further still, the effective dose of High Delta (9)-tetrahydrocannabinol (THC) Cannabis Plant Material may be administered to a subject on a high fat diet. Furthermore, the high fat diet may comprise at least 35% of the subject's total daily calories comprise fat. Still again, the effective dose of High Delta (9)-tetrahydrocannabinol (THC) Cannabis Plant Material may lower an amount of at least one CD11b+Ly6g+ monocyte in a subject. Again still, the effective dose of High Delta (9)-tetrahydrocannabinol (THC) Cannabis Plant Material may lower an amount of at least one CD11c+MHCII+ M1 pro-inflammatory macrophages in a subject.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 shows graphs illustrating at: (A) Fasting glucose (picograms/deciliter) was taken prior to treatment and after the cessation of treatment prior to euthanasia; (B) Fasting serum was collected for insulin analysis immediately prior to euthanasia. Insulin value shown as micrograms/Liter (μg/L); and (C) Calculated Homeostatic Model Assessment for Insulin Resistance (HOMA-IR) using fasting glucose and insulin.

FIG. 2 shows one embodiment of a study design of the current disclosure.

FIG. 3 shows Cannabis Effects on Body Composition and HOMA-IR.

FIG. 4 shows Cannabis Effects on Adipose Tissue Macrophages.

FIG. 5 shows Cannabis Effects on Liver Tissue Macrophages.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping but rather should be read as “and/or” unless expressly stated otherwise. Similarly, a group of items linked with the conjunction “or” should not be read as requiring mutual exclusivity among that group but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y'. The range can also be expressed as an upper limit, e.g. ‘about x, y, z, or less'and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’and ‘y’are numerical values, includes “about ‘x’to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein “cancer” can refer to one or more types of cancer including, but not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basa cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, Hairy cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodyspastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sézary syndrome, skin cancer, small intestine cancer, large intestine cancer (colon cancer), soft tissue sarcoma, T-cell lymphoma, throat cancer, oropharyngeal cancer, nasopharyngeal cancer, hypoharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor.

As used herein, “control” can refer to an alternative subject or sample used in an experiment for comparison purpose and included to minimize or distinguish the effect of variables other than an independent variable.

The term “optional” or “optionally” means that the subsequent described event, circumstance or substituent may or may not occur, and that the description includes instances where the event or circumstance occurs and instances where it does not.

As used herein, “dose,” “unit dose,” or “dosage” can refer to physically discrete units suitable for use in a subject, each unit containing a predetermined quantity of a pharmaceutical formulation thereof calculated to produce the desired response or responses in association with its administration.

The term “molecular weight”, as used herein, can generally refer to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “polymer” refers to molecules made up of monomers repeat units linked together. “Polymers” are understood to include, but are not limited to, homopolymers, copolymers, such as for example, block, graft, random and alternating copolymers, terpolymers, etc. and blends and modifications thereof. “A polymer” can be can be a three-dimensional network (e.g. the repeat units are linked together left and right, front and back, up and down), a two-dimensional network (e.g. the repeat units are linked together left, right, up, and down in a sheet form), or a one-dimensional network (e.g. the repeat units are linked left and right to form a chain). “Polymers” can be composed, natural monomers or synthetic monomers and combinations thereof. The polymers can be biologic (e.g. the monomers are biologically important (e.g. an amino acid), natural, or synthetic.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof. The effect can be therapeutic in terms of a partial or complete cure of a disease, condition, symptom or adverse effect attributed to the disease, disorder, or condition. The term “treatment” as used herein covers any treatment of cancer in a subject, particularly a human and/or companion animal, and can include any one or more of the following: (a) preventing the disease or damage from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, “water-soluble”, as used herein, generally means at least about 10 g of a substance is soluble in 1 L of water, i.e., at neutral pH, at 25° C.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Kits

Any of the compounds and/or formulations described herein can be presented as a combination kit. As used herein, the terms “combination kit” or “kit of parts” refers to the compounds, compositions, formulations, particles, and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agent(s)) contained in the kit are administered simultaneously, the combination kit can contain the active agent(s) in a single formulation, such as a pharmaceutical formulation, (e.g., a tablet, liquid preparation, dehydrated preparation, etc.) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds and/or formulations, safety information regarding the content of the compounds and formulations (e.g., pharmaceutical formulations), information regarding the dosages, indications for use, and/or recommended treatment regimen(s) for the compound(s) and/or pharmaceutical formulations contained therein. In some embodiments, the instructions can provide directions and protocols for administering the compounds and/or formulations described herein to a subject in need thereof. In some embodiments, the instructions can provide one or more embodiments of the methods for administration of Cannabis Plant Material and/or pharmaceutical formulation thereof such as any of the methods described in greater detail elsewhere herein.

Much of the mortality attributed to obesity is due to the metabolic disorders of insulin resistance, glucose intolerance, dyslipdemia, and hypertension: collectively known as metabolic syndrome. Chronic inflammation appears to function as a common etiologic mechanism driving these metabolic disorders. Indeed, inflammation is at the heart of obesity-related conditions. Studies completed over the past several decades have highlighted the central role of tissue-associated macrophages, and in particular adipose tissue and liver macrophages, in coordinating both the metabolic and inflammatory aspects of metabolic syndrome. Certainly, macrophages play a critical role in lipid storage and utilization in adipose tissue, tightly regulating metabolic health. Studies have documented that macrophages can promote metabolic pathologies such as insulin resistance. Further, macrophages can release mediators that increase hepatic triglyceride levels. In addition, macrophages can enhance formation of hepatic cholesterol crystals as well as enhance hepatic inflammation, fibrosis, and lipid oxidation. Taken together, strong evidence suggests that macrophage activation and subsequent inflammation represents a common pathogenic mechanism underlying obesity-associated insulin resistance and inflammatory diseases related to metabolic syndrome such as liver steatosis. Thus, they are ideal therapeutic targets for diseases such as insulin resistance, non-alcoholic fatty liver disease, and atherosclerosis.

Natural products have been the source of many drugs, including 60% of cancer drugs and 75% of infectious disease drugs. Humans have used natural products and their derived molecules to treat disorders for at least 60,000 years. Cannabidiol (CBD) and tetrahydrocannabinol (THC), are the two most well-known and researched cannabinoids. Despite the controversy surrounding the use of cannabis, a controlled substance, studies on health benefits are emerging. The current disclosure are proposing its use as a treatment for obesity and associated metabolic syndrome. The current disclosure provides data to document on the benefits of cannabis on metabolic syndrome including on the Homeostasis Model Assessment of Insulin Resistance (HOMA-IR), a measure used to assess insulin resistance in patients with type 2 diabetes and other populations, and on macrophages in both the adipose tissue and liver in obese mice given their importance in the development of metabolic syndrome. The current disclosure proposes to develop cannabis as an agent that can be used to treat metabolic syndrome. A treatment to prevent/reduce obesity and associated metabolic perturbations would have enormous public health implications.

A symptom of metabolic syndrome is insulin resistance, defined as the inability of insulin to optimally stimulate the transport of glucose into the body's cell (hyperinsulinemia or impaired glucose tolerance). This can lead to type II Diabetes and Cardiovascular Disease. The current disclosure has discovered that the Cannabis Plant Material can reduce fasting glucose and insulin and lower the homeostasis model assessment-estimated insulin resistance (HOMA-IR) index. Our innovation can be used to mitigate metabolic syndrome and associated Type II Diabetes, Coronary Heart Disease, Stroke.

While there are a number of medications on the market to improve insulin sensitivity in individuals with metabolic syndrome, these medications come with side effects, are expensive, and are not always effective. The current disclosure has discovered that the Cannabis Plant Material can reduce fasting glucose and insulin and lower the homeostasis model assessment-estimated insulin resistance (HOMA-IR) index in mice with obesity. The inventors obtained the Cannabis material from the National Center for Natural Products Research at the University of Mississippi.

The inventors requested Cannabis Plant Material as follows (1) Placebo Cannabis Plant Material, (2) High Delta (9)-tetrahydrocannabinol (THC) Cannabis Plant Material, and (3) High Cannabidiol (CBD) Cannabis Plant Material. Obese mice were administered one of the 3 treatments 3 times per week via gavage (administered in oil following heating of the plant) for the duration of 5 weeks. Following the treatment period, blood was collected for assessment of fasting glucose and insulin from which the homeostasis model assessment-estimated insulin resistance (HOMA-IR) index was calculated. Our data documents that High Delta (9)-tetrahydrocannabinol (THC) Cannabis Plant Material significantly reduces fasting glucose and fasting insulin and lowers the HOMA-IR indicative of improved insulin sensitivity. Our innovation can be developed as an agent to successfully mitigate metabolic syndrome.

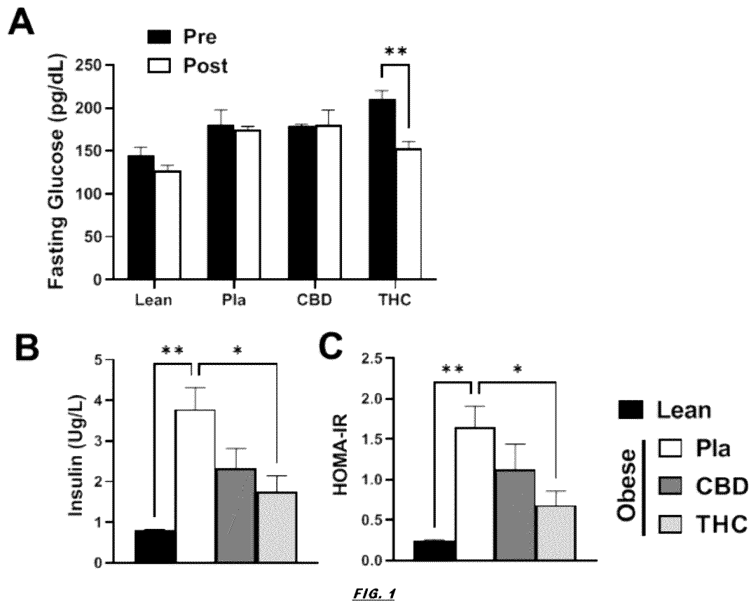

FIG. 1 shows that THC improves fasting glucose and insulin following high-fat-diet induced obesity. Mice were fed a high fat diet for 20 weeks prior to treatment. Cannabis plant material containing high (~20% w/w) amounts of Δ9-tetrahydrocannabinol (THC), high (~10% w/w) amount of cannabidiol (CBD), or a placebo very low (<0.1% w/w) THC and CBD (Pla) was suspended in corn oil and administered p.o. at 40 mg/kg (raw plant material). Treatment was administered 3×/week for 5 weeks. A) Fasting glucose (picograms/deciliter) was taken prior to treatment and after the cessation of treatment prior to euthanasia. B) Fasting serum was collected for insulin analysis immediately prior to euthanasia. Insulin value shown as micrograms/Liter (μg/L). C) Calculated Homeostatic Model Assessment for Insulin Resistance (HOMA-IR) using fasting glucose and insulin ([Insulin*Glucose]/405). Statistics: A) Repeated measures two-way ANOVA, Sidak post hoc multiple comparisons. B-C) One-way ANOVA, Tukey post hoc multiple comparisons. *Significantly different groups. p<0.05

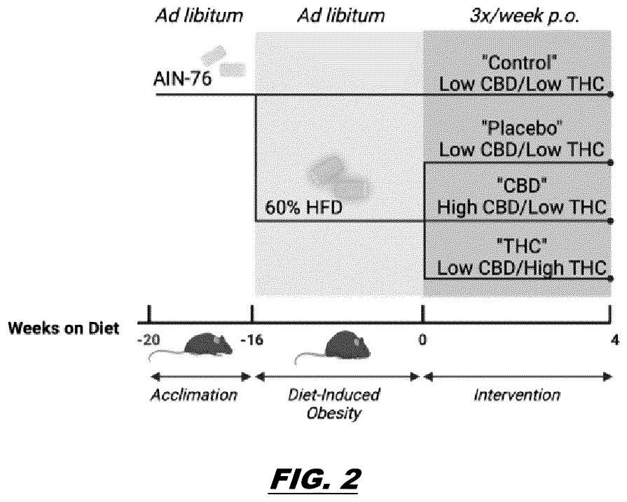

Following 4 weeks on the purified diet, mice were randomized to either stay on the low-fat diet (LFD) or switch to a high-fat diet (HFD) for the duration of 16 weeks for the development of an obesity phenotype. Following 16 weeks on the HFD, mice were further randomized to cannabis with low CBD and low THC (Placebo), cannabis with high CBD and low THC (CBD), or cannabis with high THC and low CBD (THC) for a period of 4 weeks. Mice were administered their respective cannabis treatment via oral gavage at a dose of 40mg/Kg and for 3 times per week. The current disclosure selected the 40 mg/kg dose for mice as it falls in the recommended medical amount of THC for humans; it has been recommended to start patients on daily THC levels of 2.5-5 mg daily doses (this is within our low THC treatment range) and to titrate to daily levels of 30-40 mg (this is within the high THC range). During this time, mice were maintained on their HFD. The LFD group continued on their LFD during this time and served as a LFD control reference for the study. FIG. 2 shows on arriving at the animal facility, all mice were placed on an AIN-76A purified diet.

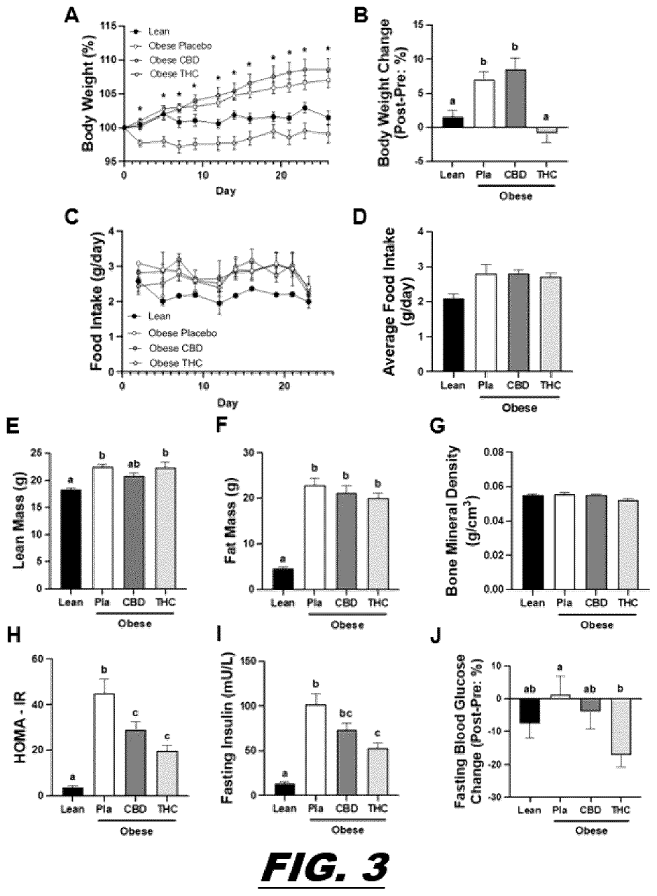

There is a significant increase in % body weight gain with Placebo and CBD over the 4-week intervention period compared to the Lean (LFD) group (FIG. 3 at A; P<0.05). Interestingly however, the THC group did not gain weight during this period despite being maintained on a HFD (FIG. 3 at A). The current disclosure examined percent body weight change (pre-and post-4 week intervention period) and consistent with the % body weight gain data in FIG. 3 at A, both the Placebo and CBD groups continued to gain weight during the 4 week intervention period compared to the Lean (LFD) control group whereas the THC group does not gain weight and is similar to the lean (LFD group) in weight change during the intervention period (FIG. 3 at B; P<0.05). Together these data show that there is no effect of high CBD on weight gain with HFD whereas high THC prevents any further weight gain on a HFD. This benefit of no further weight gain with THC consumption was independent of any changes in food intake as there were no differences in food intake across the groups (FIG. 3 at C). To further examine weight changes, current disclosure performed body composition analysis to confirm that this reduction in weight gain was not reflective of any changes in lean mass or bone mineral density. As expected, all HFD groups had increased lean mass and increased fat mass (FIG. 3 AT E-F; P<0.05). However, there was no effect of THC or CBD on lean mass (FIG. 3 at E) or bone mineral density (FIG. 3 at F) documenting preservation of lean mass and bone mineral density with CBD and THC consumption. The current disclosure assessed the Homeostasis Model Assessment of Insulin Resistance (HOMA-IR), a measure used to assess insulin resistance in patients with type 2 diabetes and other populations and show that HOMA-IR is increased with obesity as expected (FIG. 3 at H; P<0.05). However, both the CBD and THC treatments result in a dramatic reduction in the HOMA-IR (greater than 50% for THC) compared to the Placebo group (FIG. 3 at H; P<0.05). This is reflected by a decrease in fasting insulin that was particularly evident in the THC group (FIG. 3 at I; P<0.05) and a change in fasting glucose from pre-intervention levels that is again evident in the THC group (FIG. 3 at I; P<0.05) with smaller changes observed in the CBD group. Together, these data document that both CBD and THC can improve HOMA-IR with the most pronounced effects seen with THC. Further, THC but not CBD can prevent further weight gain in mice fed a HFD despite no change in food intake. FIG. 3 shows that body weight is increased with obesity as expected.

In one instance, a high fat diet may be considered to be one where at least 35% of a person's total daily calories come from fat, including both saturated and unsaturated fats; this means a significant portion of their diet consists of fatty foods like meat, butter, cheese, and oils. Further, a high-fat diet may typically contain about 32-60 % of calories from fat.

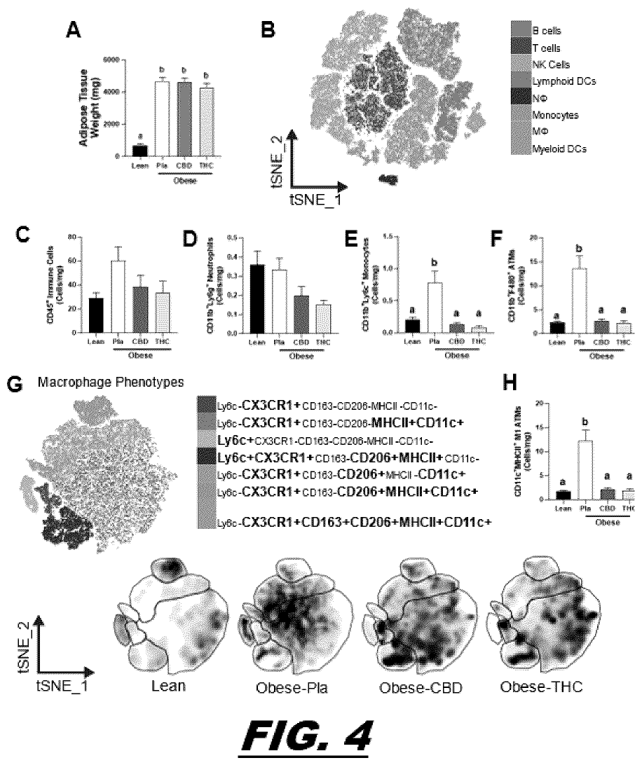

The current disclosure first assessed adipose tissue (fat pad) weight at the time of euthanasia and as expected there was an increase in adipose tissue weight in obese mice (HFD groups) compared to lean mice (LFD control group) (FIG. 4 at A). The tSNE shows the high dimensional flow cytometry data allowing for identification of distinct cell populations (FIG. 4 at B). The current disclosure first assessed all immune cells (i.e. CD45+ cells) and noted no difference in total immune cells per mg of tissue (FIG. 4 at C). For CD11b+Ly6g+ neutrophils, there were no differences across groups (FIG. 4 at D). However, for CD11b+Ly6g+ monocytes (precursors to macrophages) the current disclosure documents an increase with obesity (Placebo) that is mitigated by both CBD and THC (FIG. 4 at E; P<0.05). In fact, levels of CD11b+Ly6g+ monocytes in CBD and THC were reduced to the levels of a lean (LFD) mouse. These cells are known to contribute to inflammation, particularly in conditions like obesity, through mechanisms like releasing pro-inflammatory cytokines and promoting tissue infiltration by other immune cells, potentially exacerbating insulin resistance. For CD11b+F480+ adipose tissue macrophages followed a very similar trend with a marked increase with obesity (Placebo) that is mitigated by both CBD and THC (FIG. 4 at F; P<0.05). Given this dramatic effect on overall macrophages, the current disclosure further interrogated macrophage populations to determine effects on exact macrophage phenotypes (FIG. 4 at G). The density plots allow for comparison across groups. Of particular importance is the dramatic changes in CD11c+MHCII+ M1 pro-inflammatory macrophages. Indeed, as expected the current disclosure documents a large increase in M1 macrophages with obesity (FIG. 3 at H: P<0.05). However, this response is completely rescued with CBD and THC (FIG. 4 at H; P<0.05). These findings are of particular importance to metabolic syndrome as when activated, M1 macrophages release inflammatory cytokines that disrupt normal insulin signaling pathways within cells, leading to impaired glucose uptake and insulin resistance. These findings are consistent with the documented beneficial effects of CBD and THC on HOMA-IR. FIG. 4 shows, given the role of macrophages on insulin resistance, the current disclosure assessed the signature of adipose tissue macrophages following CBD and THC treatment in obese mice.

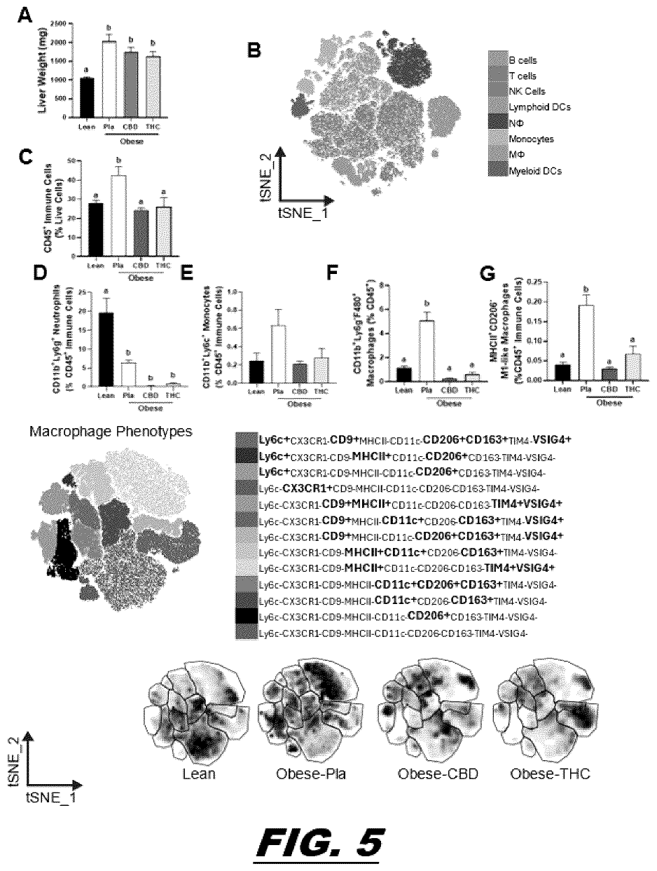

The current disclosure first assessed liver weight at the time of euthanasia and as expected there was an increase in liver weight in obese mice (HFD groups) compared to lean mice (LFD control group) (FIG. 5 at A). The tSNE shows the high dimensional flow cytometry data allowing for identification of distinct cell populations (FIG. 5 at B). The current disclosure first assessed all immune cells (i.e. CD45+ cells) and noted that there was an increase in total immune cells in obese mice (Placebo) compared to Lean mice (LFD) (FIG. 4C; P<0.05) but this was rescued with CBD and THC (FIG. 5 at C; P<0.05); there was no difference in total immune cells between Lean mice (LFD) and Obese CBD or Obese THC mice. For CD11b+Ly6g+ neutrophils, there was a decrease in this immune cell population with obesity (FIG. 5 at D; P<0.05) but no differences across obese groups. For CD11b+Ly6g+ monocytes (precursors to macrophages) there was an increase with obesity (Placebo) that appeared to be mitigated by both CBD and THC (FIG. 4 at E) but this did not reach statistical significance. For CD11b+F480+ liver tissue macrophages there was a marked increase with obesity (Placebo) that was rescued by both CBD and THC (FIG. 5 at F; P<0.05). Given this dramatic effect on overall macrophages, the current disclosure further interrogated macrophage populations to determine effects on exact macrophage phenotypes. The density plots allow for comparison across groups. Of particular importance is the dramatic changes in MHCII+CD206− M1 pro-inflammatory macrophages. Indeed, as expected the current disclosure documented a large increase in M1 macrophages with obesity (FIG. 5 at G: P<0.05). However, this response is completely rescued with CBD and THC (FIG. 4 at G; P<0.05). These findings are of particular importance to metabolic syndrome as when activated, M1 macrophages release inflammatory cytokines that disrupt normal insulin signaling pathways within cells, leading to impaired glucose uptake and insulin resistance. These findings are consistent with the documented beneficial effects of CBD and THC on HOMA-IR. FIG. 5 shows, given the role of macrophages on insulin resistance and the fact that the liver is a metabolic organ, the current disclosure assessed the signature of liver tissue macrophages following CBD and THC treatment in obese mice.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A therapeutic method for treating metabolic syndrome comprising:
administering an effective dose of High Delta (9)-tetrahydrocannabinol (THC) Cannabis Plant Material to a subject;
wherein the effective dose reduces a fasting glucose level and a fasting insulin level and lowers a homeostasis model assessment-estimated insulin resistance (HOMA-IR) index in the subject; and
wherein the effective dose of High Delta (9)-tetrahydrocannabinol (THC) Cannabis Plant Material increase insulin sensitivity in the subject.

2. The therapeutic method for treating metabolic syndrome of claim 1, wherein the effective dose of High Delta (9)-tetrahydrocannabinol (THC) Cannabis Plant Material administered to the subject in an increasing dosage.

3. The therapeutic method for treating metabolic syndrome of claim 1, wherein the effective dose of High Delta (9)-tetrahydrocannabinol (THC) Cannabis Plant Material is administered in a dosage of 30 to 40 mg.

4. The therapeutic method for treating metabolic syndrome of claim 1, wherein the effective dose of High Delta (9)-tetrahydrocannabinol (THC) Cannabis Plant Material is administered daily.

5. The therapeutic method for treating metabolic syndrome of claim 1, wherein the effective dose of High Delta (9)-tetrahydrocannabinol (THC) Cannabis Plant Material is administered to a subject on a high fat diet.

6. The therapeutic method for treating metabolic syndrome of claim 5, wherein the high fat diet includes at least 35% of the subject's total daily calories comprising fat.

7. The therapeutic method for treating metabolic syndrome of claim 1, wherein the effective dose of High Delta (9)-tetrahydrocannabinol (THC) Cannabis Plant Material lowers an amount of at least one CD11b+Ly6g+ monocyte in a subject.

8. The therapeutic method for treating metabolic syndrome of claim 1, wherein the effective dose of High Delta (9)-tetrahydrocannabinol (THC) Cannabis Plant Material lowers an amount of at least one CD11c+MHCII+ M1 pro-inflammatory macrophages in a subject.

9. A treatment for reducing obesity and associated metabolic perturbations comprising:
administering an effective dose of High Delta (9)-tetrahydrocannabinol (THC) Cannabis Plant Material to a subject;
wherein the effective dose reduces a fasting glucose level and a fasting insulin level and lowers a homeostasis model assessment-estimated insulin resistance (HOMA-IR) index in the subject; and
wherein the effective dose of High Delta (9)-tetrahydrocannabinol (THC) Cannabis Plant Material increase insulin sensitivity in the subject.

10. The therapeutic method for treating metabolic syndrome of claim 9, wherein the effective dose of High Delta (9)-tetrahydrocannabinol (THC) Cannabis Plant Material administered to the subject in an increasing dosage.

11. The treatment for reducing obesity and associated metabolic perturbations of claim 9, wherein the effective dose of High Delta (9)-tetrahydrocannabinol (THC) Cannabis Plant Material is administered in a dosage of 30 to 40 mg.

12. The treatment for reducing obesity and associated metabolic perturbations of claim 9, wherein the effective dose of High Delta (9)-tetrahydrocannabinol (THC) Cannabis Plant Material is administered daily.

13. The treatment for reducing obesity and associated metabolic perturbations of claim 9, wherein the effective dose of High Delta (9)-tetrahydrocannabinol (THC) Cannabis Plant Material is administered to a subject on a high fat diet.

14. The t treatment for reducing obesity and associated metabolic perturbations of claim 13, wherein the high fat diet includes at least 35% of the subject's total daily calories comprising fat.

15. The treatment for reducing obesity and associated metabolic perturbations of claim 9, wherein the effective dose of High Delta (9)-tetrahydrocannabinol (THC) Cannabis Plant Material lowers an amount of at least one CD11b+Ly6g+ monocyte in a subject.

16. The treatment for reducing obesity and associated metabolic perturbations of claim 9, wherein the effective dose of High Delta (9)-tetrahydrocannabinol (THC) Cannabis Plant Material lowers an amount of at least one CD11c+MHCII+ M1 pro-inflammatory macrophages in a subject.
